SEQUENCE LISTING (13 sequences)

SEQ ID NO: 1 - protein, 6 aa, artificial sequence
  synthetic construct
QWAVGH

SEQ ID NO: 2 - protein, 7 aa, artificial sequence
  synthetic construct
  SITE (1)..(1): D-phenylalanine
FQWAVGH

SEQ ID NO: 3 - protein, 7 aa, artificial sequence
  synthetic construct
  SITE (7)..(7): statin
QWAVGHX

SEQ ID NO: 4 - protein, 8 aa, artificial sequence
  synthetic construct
  SITE (1)..(1): D-phenylalanine
  SITE (8)..(8): statin
FQWAVGHX

SEQ ID NO: 5 - protein, 7 aa, artificial sequence
  synthetic construct
  MOD_RES (7)..(7): Leu(PSI)(CHOH-CH2)-(CH2)2-CH3
QWAVGHL

SEQ ID NO: 6 - protein, 7 aa, artificial sequence
  synthetic construct
  SITE (1)..(1): D-phenylalanine
  MOD_RES (7)..(7): Leu(PSI)(CHOH-CH2)-(CH2)2-CH3
FQWAVGH

SEQ ID NO: 7 - protein, 8 aa, artificial sequence
  synthetic construct
  SITE (1)..(1): D-phenylalanine
  MOD_RES (8)..(8): Leu(PSI)(CH2NH)
FQWAVGHL

SEQ ID NO: 8 - protein, 7 aa, artificial sequence
  synthetic construct
  MOD_RES (7)..(7): Leu(PSI)(CH2NH)
QWAVGHL

SEQ ID NO: 9 - protein, 7 aa, artificial sequence
  synthetic construct
  SITE (6)..(6): statin
  MOD_RES (7)..(7): C-terminal amidation
QWAVGHL

SEQ ID NO: 10 - protein, 8 aa, Artificial Sequence
  synthetic construct
  SITE (1)..(1): D-phenylalanine
  SITE (7)..(7): statin
  MOD_RES (8)..(8): C-terminal amidation
FQWAVGHL

SEQ ID NO: 11 - protein, 8 aa, Artificial Sequence
  synthetic construct
  SITE (7)..(7): statin
  MOD_RES (8)..(8): C--terminal amidation
FQWAVGHL

SEQ ID NO: 12 - protein, 8 aa, Artificial Sequence
  synthetic construct
  SITE (7)..(7): statin
  MOD_RES (8)..(8): C-terminal amidation
FQWAVGHL

SEQ ID NO: 13 - protein, 8 aa, Artificial Sequence
  synthetic construct
  SITE (7)..(7): statin
  MOD_RES (8)..(8): C-terminal amidation
FQWAVGHL

(19) United States
(12) Patent Application Publication
(10) Pub. No.: US 2025/0319214 A1
(43) Pub. Date: Oct. 16, 2025

(54) A NOVEL KIT FOR RADIOPHARMACEUTICAL PREPARATION OF A RADIOMETAL LABELED CHELATE-FUNCTIONALIZED TARGETING CONJUGATE

(71) Applicant: Life Molecular Imaging Ltd., Warwick (GB)

(72) Inventors: Marion ZERNA, Berlin (DE); Hanno SCHIEFERSTEIN, Aarbergen (DE); Mathias BERNDT, Berlin (DE)

(73) Assignee: Life Molecular Imaging Ltd., Warwick (GB)

(21) Appl. No.: 18/574,445

(22) PCT Filed: Jun. 29, 2022
(86) PCT No.: PCT/EP2022/067985

§ 371 Date: Dec. 27, 2023

(30) Foreign Application Priority Data
Jun. 29, 2021 (EP) 21182551.8

(51) Int. Cl. A61K 51/08; A61K 9/19

(52) U.S. Cl. CPC A61K 51/088; A61K 9/19; A61K 2121/00

(57) ABSTRACT

Subject matter of the present invention is a lyophilized kit formulation for the preparation of radiometal labeled chelate-functionalized GRP receptor targeting conjugates comprising
a chelate-functionalized GRP receptor targeting conjugate comprising
i. a chelating moiety
ii. at least one targeting moiety, wherein said targeting moiety is a GRP receptor (GRPr) targeting peptide, and
iii. optionally, at least one linker, connecting the chelating moiety with the GRP receptor (GRPr) targeting moiety, and
at least one GRP receptor (GRPr) targeting moiety,
at least one non-reducing sugar selected from the group comprising trehalose and sucrose, and
at least one radio stabilizer, selected from the group comprising ascorbic acid, ascorbic acid salts, gentisic acid, gentisic acid salts, or mixtures thereof.

The present invention is directed towards a lyophilized kit formulation for the preparation of radiometal labeled chelate-functionalized targeting conjugates comprising:
a chelate-functionalized targeting conjugate,
at least one non-reducing sugar selected from the group comprising trehalose and sucrose
at least one radio stabilizer, selected from the group comprising ascorbic acid, ascorbic acid salts, gentisic acid, gentisic acid salts, or mixtures thereof.

### PRIOR ART

The use of Kit Formulations for preparation of radiometal labeled peptides, in particular ⁶⁸Ga-labeled peptides have been described in the prior art:

WO2016030103 (Wouters et. al) discloses a radiolabeling kit.

The kit comprises a suitable amount of an acetate salt or buffer, a chelate functionalized targeting agent and a metal inhibitor, which is a co-chelating agent, capable of inactivating contaminating metals.

The application is not solving the problem of long-term stability of Kit Formulations during storage.

WO2016030104 (Wouters et. al) discloses radiometal labeling methods.

The method comprises the use of a “metal inhibitor” during the radiolabeling reaction to increase the radiolabeling yield of the radiometal labeled chelate functionalized targeting agent.

The application is not solving the problem of long-term stability of Kit Formulations during storage.

Nassiri et al. (Nassiri et al.: coalitionforpetdrugapproval.files.wordpress.com/2016/06/nassiri-paulus-kit-for-ga-68-dotatate.pdf) discloses a kit for the Preparation of gallium Ga 68 dotatate.

The kit comprises the somatostatin analog dotatate, phenanthroline (as metal sequesting agent), gentisic acid and mannitol. The kit has a shelf life of 12 months at room temperature.

Pandey et al. (J Radioanal Nucl Chem, 2016, 1115-1124) describe single vial AMBA kit for ⁶⁸Ga labeling. The lyophilized Kit formulation contains AMBA peptide, ascorbic acid and sodium acetate. The Kits are stored at −20° C. until further use.

de Barros et al. (Appl. Rad. and Isotopes, 2012, 1440-2445) describe a Kit formulation for pratapartion of 99mTc-labeled HYNIC-bAla-Bombesin(7-14). The Kit formulation comprises stannous chloride for ^{99m}Tc-labeling. The stability of the Kit formulations was confirmed at −20°.

Vats et al. (Journal of Pharmaceutical and Biomedical Analysis, 2019, 39-44) describe a kit containing the peptide and sodium acetate.

The problem to be solved by the current invention is to provide a lyophilized Kit Formulation for preparation of a radiometal labeled chelate-functionalized targeting conjugate, in particular for a radiometal labeled chelate-functionalized GRP receptor (GRPr) targeting conjugate, that:
Can be easily used for the preparation for the radiometal labeled chelate-functionalized targeting conjugate.
Are used with generator or cyclotron derived solutions of the radiometal without reduction of the radiometal during the preparation step.
For labeling with ⁶⁸Ga can be used with various generators (and elution method, e.g. volume and concentration of HCl).
Provides the radiometal labeled chelate-functionalized targeting conjugate without or minor decomposition by radiolysis.
Provides the radiometal labeled chelate-functionalized targeting conjugate in high purity of not less than 90%, preferably, not less than 92%, more preferably not less than 95%.
Meets regulatory requirements for lyophilization of parenterals including dose uniformity and stability, cake and the cake appearance.
Can be stored at room temperature over a period of at least one year without decomposition of the chelate functionalized chelate-functionalized targeting conjugate (purity of the chelate functionalized peptide conjugate not less than 90%, preferably not less than 95%, more preferably not less than 98%).

Nassiri et al. and WO2016030103 (Wouters et. al) teach the use of sugar alcohols (mannitol) as an excipient in the kit formulation for a chelate functionalized peptide to obtain a composition that is stable for 12 months at room temperature and that can be easily labeled with ⁶⁸Ga isotope. Despite of this teaching, sugar alcohol mannitol was found to be insufficient for the manufacturing of a lyophilized Kit Formulation comprising a chelate functionalized GRPr targeting peptide.

Pandey et al. describe a single vial Kit. Due to the pre-defined amount of buffer (sodium acetate) present in the Kit, the use of the Kit is not flexible towards various generators (variation of HCl volume and concentration). The storage of those Kits is at −20° C. In addition, the sodium acetate present is not a suitable excipient for lyophilization.

The kit described by Vats et al. contains no radioscavenger that would be required to stabilize the radiometal labeled chelate-functionalized targeting conjugate especially at higher radioactivity levels. Furthermore, the kit contains sodium acetate. Due the glass temperature of the components a lyophilization according to regulatory requirements for lyophilization of parenterals including dose uniformity and stability can not be achieved.

In contrast to the use of mannitol, a sugar alcohol, Kit Formulations containing non-reducing sugars were found to meet the requirements for lyophilized Kit Formulations, in particular:
Easy preparation for the radiometal labeled chelate-functionalized targeting conjugate.
High purity of the radiometal labeled chelate-functionalized targeting conjugate.
Meets regulatory requirements for lyophilization of parenterals including dose uniformity and stability.
High stability during storage at room temperature.

The present invention concerns an improved method of radiopharmaceutical preparation of radiometal labeled chelate-functionalized targeting conjugate by using a specific kit formulation.

### DETAILED DESCRIPTION

Unless otherwise defined, all terms of art, notations and other scientific terminology used herein are intended to have the meanings commonly understood by those of ordinary skill in the art to which the present application pertains. In some cases, terms with commonly understood meanings are defined herein for clarity and/or for ready reference, and the inclusion of such definitions herein should not necessarily be construed to represent a substantial difference over what is generally understood in the art.

As used herein and in the appended claims, the singular forms “a”, “and”, and “the” include plural referents unless the context clearly dictates otherwise.

As used herein and in the appended claims, the term “agent” has the same meaning as the term “moiety” and both terms can be interchangeably replaced by each other.

The invention illustratively described herein suitably may be practiced in the absence of any element or elements, limitation or limitations that are not specifically disclosed herein. Thus, for example, in each instance herein any of the terms “comprising”, “consisting essentially of and “consisting of may be replaced with either of the other two terms.

Whereas the term “one or more”, such as one or more members of a group of members, is clear per se, by means of further exemplification, the term encompasses inter alia a reference to any one of said members, or to any two or more of said members, such as, e.g., any >3, >4, >5, >6 or >7 etc. of said members, and up to all said members.

All documents cited in the present specification are hereby incorporated by reference in their entirety.

The terms “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid metrics that function in a manner similar to the naturally occurring amino acids. Naturally occurring amino acids are those encoded by the genetic code, as well as those amino acids that are later modified, e.g., hydroxyproline, γ-carboxyglutaniate, and 0-phosphoserme. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “labeling buffer” refers to a solution that must be nontoxic, must effectively maintain the pH within a range of 3.0 to 5.0, should not compete with gallium-68 ions and have preferably a low capacity for metal chelation with regard to the capacity of the chelating agent as assembled with the targeting agent. It must also be able to tolerate possible small changes in the volume of generator eluate (and therefore the amount of HCl), i.e. it must be strong enough to maintain the pH within the desired range with 10% changes in the volume of eluate. Suitable buffers include, e.g., acetate, formate, tartrate, citrate, phosphate and the like.

By the term “radiopharmaceutical preparation” or “radiopharmaceutical composition” is meant a composition comprising the radiometal complex of the invention in a form suitable for human administration. For human administration a radiopharamceutical preparation must be sterile. Alternatively, the radiopharmaceutical preparation of the present invention may also be provided in a unit dose form ready for human injection and could for example be supplied pre-filled sterile syringe. The syringe containing the unit dose would also be supplied within a syringe shield (to protect the operator from potential radioactive dose).

In the following passages, different aspects or embodiments of the invention are defined in more detail. Every aspect or embodiment so defined may be combined with each of the other aspects or embodiments unless stated otherwise. In particular, any feature indicated as being preferred or advantageous in one embodiment may be combined with any other embodiment or embodiments indicated as being preferred or advantageous.

Subject matter of the present invention is a lyophilized kit formulation for the preparation of radiometal labeled chelate-functionalized targeting conjugates comprising:
a chelate-functionalized targeting conjugate,
at least one non-reducing sugar selected from the group comprising trehalose and sucrose.
at least one radio stabilizer, selected from the group comprising ascorbic acid, ascorbic acid salts, gentisic acid, gentisic acid salts, or mixtures thereof.

Subject matter of the present invention is a lyophilized kit formulation for the preparation of radiometal labeled chelate-functionalized targeting conjugates, wherein in particular the concentration of the non-reducing sugar in said formulation is 10-600 μmol, preferably, 20-500 μmol, more preferably, 50-300 μmol.

In one embodiment of the kit formulation of the present invention, said formulation contains 5-250 mg, preferably 10-100 mg trehalose.

In one embodiment of the kit formulation of the present invention, said formulation contains 5-250 mg, preferably 10-100 mg sucrose.

In another embodiment of the kit formulation a mixture of non-reducing sugars is used.

In one embodiment of the kit formulation of the present invention, said radiostabilizer is selected from the group comprising ascorbic acid, ascorbic acid salts, or mixtures thereof.

In one specific embodiment of the kit formulation of the present invention, said radiostabilizer is ascorbic acid.

In one embodiment of the kit formulation of the present invention, the concentration of said radiostabilizer in the formulation is 1-500 μmol, preferably 5-250 μmol, more preferably 10-100 μmol.

In one embodiment of the kit formulation of the present invention, the amount of ascorbic acid in the formulation is 1-20 mg, more preferably 1-10 mg.

In one embodiment of the kit formulation of the present invention, the chelate functionalized targeting conjugate comprises:
a chelating agent or moiety,
optionally, at least one linker, connecting the chelating moiety with the targeting moiety, and
at least one targeting agent or moiety.

As used herein, a “chelate-functionalized targeting conjugate” refers to a targeting agent/moiety capable of being labeled with a radioisotope such as for example gallium-68, by means of a chelating agent/moiety which is linked to the targeting agent. Optionally, at least one linker is present to connect the chelating agent/moiety with the targeting agent/moiety.

Preferred chelating agents for functionalizing a targeting agent to be radiolabeled with gallium-68 are those which form stable chelates with Ga³⁺, in particular ⁶⁸Ga³⁺ (the radioisotope generator eluted from a germanium-68/gallium-68 generator using HCl), at least for a time sufficient for diagnostic investigations using such radiolabeled targeting agents. Suitable chelating agents include aliphatic amines, linear or macrocyclic such as macrocyclic amines with tertiary amines.

While these examples of suitable chelating agents are not limited, they preferably include the DOTA, NOTA and its derivatives, such as TACN, TACN-TM, DTAC, H3NOKA, NODASA, NODAGA, NOTP, NOTPME, PrP9, TRAP, Trappist Pr, NOPO, TETA; Tris(hydroxypyridinone) (THP) and derivatives, chelates open chain such as HBED, DFO or desferrioxamine or desferal, EDTA, 6SS, B6SS, PLED, TAME, YM103; NTP (PRHP)3; the H2dedpa and its derivatives such as H2dedpa-1, 2-H2dedpa, H2dp-bb-NCS, and H2dp-N-NCS; (4,6-Me02sal) 2-BAPEN; and citrate and derivatives thereof. In one embodiment of the kit formulation of the present invention, said chelating moiety is selected from the group comprising NOTA and its derivatives and/or DOTA and its derivatives. In one specific embodiment of the kit formulation of the present invention, the chelating moiety is DOTA.

The targeting agent can be a peptide, for example, a peptide comprising 2 to 20 amino acids, a polypeptide, a protein, a vitamin, a saccharide, for example a monosaccharide or a polysaccharide, an antibody and its derivatives such as nanobodies, diabodies, antibodies fragments, nucleic acid, an aptamer, an antisense oligonucleotide, an organic molecule, or any other biomolecule that is able to bind to a certain diagnostic target or to express a certain metabolic activity.

Targeting agents as described herein preferably have a capacity of biological targeting. Non-limiting examples of suitable targeting agents include molecules that target VEGF receptors, analogs of bombesin or GRP receptor (GRPr) targeting molecules, molecules targeting somatostatin receptors, RGD peptides or molecules targeting αvβ3 and αvβ5, annexin V or molecules targeting the apoptotic process, molecules targeting estrogen receptors. More generally, a list targeting molecules, organic or not, functionalized by a chelating agent can be found in the journal of Velikyan et al., Theranostic 2014, Vol. 4, Issue 1 “Prospective of 68Ga-Radiopharmaceutical Development.”

The peptides of the present invention may be naturally occurring or synthetic origin, but are preferably synthetic.

In one embodiment of the kit formulation of the present invention, said targeting moiety of the chelate functionalized peptide conjugate comprises GRP receptor (GRPr) targeting molecules, more preferably GRPr targeting peptide sequences selected from the group comprising:

Gln-Trp-Ala-Val-Gly-His,
D-Phe-Gln-Trp-Ala-Val-Gly-His,
Gln-Trp-Ala-Val-Gly-His-Sta,
D-Phe-Gln-Trp-Ala-Val-Gly-His-Sta,
Gln-Trp-Ala-Val-Gly-His-Leuψ(CHOH-CH₂)-
(CH₂)₂-CH₃,
D-Phe-Gln-Trp-Ala-Val-Gly-His-Leuψ(CHOH-
CH2)-(CH2)₂-CH₃,
D-Phe-Gln-Trp-Ala-Val-Gly-His-Leuψ(CH₂NH),
Gln-Trp-Ala-Val-Gly-His-Leuψ(CH₂NH),
Gln-Trp-Ala-Val-Gly-His-Sta-Leu-NH₂,
D-Phe-Gln-Trp-Ala-Val-Gly-His-Sta-Leu-NH₂.

In one embodiment of the kit formulation of the present invention, said chelate functionalized peptide conjugates are selected from the group comprising:
DOTA-4-amino-1-carboxymethyl-piperidine-D-Phe-Gln-Trp-Ala-Val-Gly-His-Sta-Leu-NH₂,
NOTA-4-amino-1-carboxymethyl-piperidine-D-Phe-Gln-Trp-Ala-Val-Gly-His-Sta-Leu-NH₂,
NODAGA-4-amino-1-carboxymethyl-piperidine-D-Phe-Gln-Trp-Ala-Val-Gly-His-Sta-Leu-NH₂.

In one embodiment of the kit formulation of the present invention, said chelate functionalized peptide conjugate is DOTA-4-amino-1-carboxymethyl-piperidine-D-Phe-Gln-Trp-Ala-Val-Gly-His-Sta-Leu-NH₂.

In one embodiment of the kit formulation of the present invention, at least one linker is present to connect the chelating agent/moiety with the targeting agent/moiety. The linker of the chelate functionalized chelate-functionalized targeting conjugate chelate-functionalized targeting conjugate is selected from the group comprising a bond, a natural amino acid, an unnatural amino acid, a linear diamine, a cyclic diamine, a linear carboxylic acid, a cyclic carboxylic acid, polyethylene glycol (PEG) and combinations thereof. Linker may comprise a peptide sequence of about 5 to 9 amino acids, with or without the inclusion of other groups such as aliphatic chains of up to 5 carbons in length. Preferred linker groups -poly-Lys-, -poly-Glu-, -(Gly)Z-Glu-(Lys)₃-, (Gly)₂ Glu-Lys-Glu-Lys-, (Phe)₂-(CH₂)ₛ-, (Lys)₆-Gly-, -(Gly)₃-(DGlu)₃- and -(Gly)3(aminocaproic acid)2-.

In one embodiment of the kit formulation of the present invention the linker is selected from the group comprising a natural amino acid, an unnatural amino acid, a linear diamine, a cyclic diamine, a linear carboxylic acid, a cyclic carboxylic acid, polyethylene glycol (PEG) and combinations thereof.

More preferably the linker is selected from the group comprising 4-amino-1-carboxymethylpiperidine, (R,S)-diaminoaceticacid, PEG₁₋₂₄, Sar₋₁₀, 8-aminooctanoic acid, 6-aminocaproic acid, 4-(2aminoethyl)-1-carboxymethyl piperazine, diaminobutyric acid, hippuric acid, 4-amino-1-Boc-piperidine-4-carboxylic acid, Gly-aminobenzoic acid, 5-amino-3-oxa-pentyl-succinamic acid, PEG₁₋₂₄-4-amino-1-carboxymethyl piperidine, Dab(shikimic acid), (D-Gln)x, (D-Asn)x.

In one embodiment of the kit formulation of the present invention, the linker is 4-amino-1-carboxymethyl-piperidine.

In one embodiment of the kit formulation of the present invention, said kit formulation contains 5-500 nmol of the chelate-functionalized targeting conjugate, preferably, 5-150 nmol of the chelate functionalized targeting conjugate, more preferably, 10-100 nmol of the chelate functionalized targeting conjugate.

In one embodiment of the kit formulation of the present invention the formulation in the lyophilized state can be a crystalline, a partially crystalline, partially amorphous or an amorphous formulation.

In one embodiment of the kit formulation of the present invention, the formulation is an amorphous formulation.

In one embodiment of the kit formulation of the present invention the formulation has a residual moisture of ≤1%, preferably ≤0.5%.

In one embodiment of the kit formulation of the present invention the lyophilized formulation is sterile.

In one embodiment of the kit formulation of the present invention, the decomposition of the chelate-functionalized targeting conjugate in the formulations after storage for 12 months at 25° C./60% RH is ≤10%, preferably ≤5%, more preferably ≤3%.

In one embodiment of the kit formulation of the present invention, the radiochemical purity of the radiometal labeled chelate-functionalized targeting conjugate prepared from the formulation is after storage for 12 months at 25° C./60% RH≥90%, preferably ≥93%, more preferably≥95%.

In one embodiment of the kit formulation of the present invention, the formulation for preparation of chelate-functionalized targeting conjugates is labeled with a trivalent or a bivalent radiometal cation.

In one embodiment of the kit formulation of the present invention, the formulation for preparation of chelate-functionalized targeting conjugates is labeled with a trivalent radiometal cation.

In one embodiment of the kit formulation of the present invention, the formulation for preparation of chelate-functionalized targeting conjugates labeled with a bivalent radiometal cation.

In one embodiment of the kit formulation of the present invention, the formulation for preparation of chelate-functionalized targeting conjugates labeled with an isotope is selected from the group comprising Ga, Cu, Lu, Y, Pb, Ac, Bi, Sc, Th.

In one embodiment of the kit formulation of the present invention, the formulation for preparation of chelate-functionalized targeting conjugates labeled with an isotope is selected from the group comprising ⁶⁸Ga, ⁴Cu, ⁶⁷Cu, ¹⁷⁷Lu, ⁸⁶Y, ⁹⁰Y, ²¹²Pb, ²²⁵Ac, ²¹³Bi, ⁴⁴Sc, ²²⁷Th.

In one embodiment of the kit formulation of the present invention, the formulation for preparation of chelate-functionalized targeting conjugates labeled with an isotope is selected from the group comprising ⁶⁸Ga, ⁶⁴Cu, ⁶⁷Cu, ¹⁷⁷Lu, ⁸⁶Y, ⁹⁰Y, ²¹²Pb, ²²⁵Ac, ²¹³Bi, ⁴⁴Sc, ²²⁷Th or ¹⁸F-A1.

In one embodiment of the kit formulation of the present invention, the formulation for preparation of chelate-functionalized targeting conjugates labeled with an isotope is selected from the group comprising ⁶⁸Ga, ⁶⁴Cu, ⁶⁷Cu, ¹⁷⁷Lu, ⁸⁶Y, ⁹⁰Y, ²¹²Pb, ²²⁵Ac, ²¹³Bi, ⁴⁴Sc or ¹⁸F—Al.

In one specific embodiment of the kit formulation of the present invention, the formulation for preparation of chelate-functionalized targeting conjugates is labeled with ⁶⁸Ga.

In one embodiment of the kit formulation of the present invention, the formulation for preparation of chelate-functionalized targeting conjugates is labeled with ¹⁷⁷Lu.

In one embodiment of the kit formulation of the present invention, the formulation for preparation of chelate-functionalized targeting conjugates is labeled with ¹⁸F—Al.

Subject of the present invention is also a method for preparation of a radiometal labeled chelate-functionalized targeting conjugate by using said kit formulation according to the present invention, comprising the steps of
mixing a solution of the radiometal with the kit formulation according to any one of the preceding embodiments,
optionally, adding at least one labeling buffer or a mixture of labeling buffers,
complexing the chelate-functionalized targeting conjugate according to any one of the preceding embodiments with the radiometal.

In one embodiment said method further comprises the steps of:
adding a diluent after the step of complexation, and/or
dispensing the volume of administration of the radiometal labeled chelate-functionalized targeting conjugate.

In one embodiment, the method for preparation of a radiometal labeled chelate-functionalized targeting conjugate by using said kit formulation according to the present invention, comprising the steps of
mixing a solution of the radiometal with the kit formulation according to any one of the preceding embodiments,
optionally, adding at least one labeling buffer or a mixture of labeling buffers,
complexing the chelate-functionalized targeting conjugate according to any one of the preceding embodiments with the radiometal,
adding a diluent after the step of complexation, and/or
dispensing the volume of administration of the radiometal labeled chelate-functionalized targeting conjugate.

On one embodiment the method for radiopharmaceutical preparation is conducted manually.

In another embodiment a device is used to
add the solution of the radiometal,
optionally add at least one (labeling) buffer,
conduct the complexation of the chelate-functionalized targeting conjugate, or
optionally add diluent, preferably selected from the group comprising water for injection, saline or physiological buffer.

In one particular embodiment of the method according to the invention, said complexation step is performed between 0-150° C., preferably at 25° C. (room temperature) or between 50° C. and 150° C., more preferably between 80° C. and 120° C., even more preferably between 9° and 110° C.

In one particular embodiment of the method according to the invention, for complexation, the formulation can be heated using any type of a heater or microwave.

In one particular embodiment of the method according to the invention, the complexation is performed for 0.5 min to 30 min, preferably 1 min to 20 min, preferably 1 min to 10 min, more preferably 5 to 10 min.

In one particular embodiment of the method according to the invention, said (labeling) buffer is selected from the group comprising acetate buffer, formate buffer.

In one particular embodiment of the method according to the invention, the complexation is performed at a pH value of 2.5 to 5, more preferably 3.5 to 4.5.

In one particular embodiment of the method according to the invention, the chelate-functionalized targeting conjugate obtained by this method exhibits a purity of ≥90%, preferably ≥92%, more preferably ≥95%.

The invention also concerns a radiopharmaceutical composition comprising any of the above radiometal labeled chelate-functionalized targeting conjugate of the invention.

In one embodiment, the radiopharmaceutical composition of the invention comprises:
radiometal labeled chelate-functionalized targeting conjugate of the present invention,
at least one non-reducing sugar selected from the group comprising trehalose and sucrose, and
at least one radio stabilizer, selected from the group comprising ascorbic acid, ascorbic acid salts, gentisic acid, gentisic acid salts, or mixtures thereof.

In one particular embodiment subject matter of the present invention is a kit comprising:
a vial comprising the lyophilized kit formulation according to the present invention as described above,
a vial or syringe comprising at least one labeling buffer or a mixture of labeling buffers and/or
a vial or syringe comprising a solution of the radiometal

In a preferred embodiment, the kit formulation is sterile.

In a preferred embodiment, the labeling buffer is sterile.

In a preferred embodiment, the diluent is sterile.

In a preferred embodiment the method for radiopharmaceutical preparation is providing a sterile solution of the radiometal labeled chelate-functionalized targeting conjugate. Preferably, said solution is ready for use for administration into human.

With the above context, the following consecutively numbered embodiments provide further specific aspects of the invention:

1. A lyophilized kit formulation for the preparation of radiometal labeled chelate-functionalized targeting conjugates comprising
a chelate-functionalized targeting conjugate,
at least one non-reducing sugar selected from the group comprising trehalose and sucrose.
at least one radio stabilizer, selected from the group comprising ascorbic acid, ascorbic acid salts, gentisic acid, gentisic acid salts, or mixtures thereof.

2. The kit formulation according to embodiment 1, wherein the concentration of the non-reducing sugar in said formulation is 10-600 μmol, preferably, 20-500 μmol, more preferably, 50-300 μmol.

3. The kit formulation according to embodiment 1 or 2, wherein said kit formulation contains 5-250 mg, preferably 10-100 mg trehalose.

4. The kit formulation according to embodiment 1 or 2, wherein said kit formulation contains 5-250 mg, preferably 10-100 mg sucrose.

5. The kit formulation according to any one of embodiments 1 to 4, wherein said radiostabilizer is selected from the group comprising ascorbic acid, ascorbic acid salts, or mixtures thereof.

6. The kit formulation according to any one of embodiments 1 to 5, wherein the concentration of said radiostabilizer in the formulation is 1-500 μmol, preferably 5-250 μmol, more preferably 10-100 μmol.

7. The kit formulation according to any one of embodiments 1 to 6, wherein the amount of ascorbic acid in the formulation is 1-20 mg, more preferably 1-10 mg.

8. The kit formulation according to any one of embodiments 1 to 7, wherein the chelate functionalized targeting conjugate comprises
a chelating moiety,
optionally, at least one linker, connecting the chelating moiety with the targeting moiety, and
at least one targeting moiety.

9. The kit formulation according to embodiment 8, wherein said chelating moiety is selected from the group comprising TACN, TACN-TM. DTAC, -3NOKA, NODASA, NODAGA, NOTP, NOTPME, PrP9, TRAP, Trappist Pr, NOPO, TETA; Tris(hydroxypyridinone) (THP) and derivatives, chelates open chain such as HBED, DFO or desferrioxamine or desferal, EDTA, 6SS, B6SS, PLED, TAME, YM103; NTP (PRHP)3; the H2dedpa and its derivatives such as 1-12dedpa-1, 2-12dedpa, H2dp-bb-NCS, and H2dp-N-NCS; (4,6-Me02sal) 2-BAPEN; and citrate and derivatives thereof.

10. The kit formulation according to embodiment 8, wherein the chelating moiety is NOTA and its derivatives and/or DOTA and its derivatives, preferably DOTA.

11. The kit formulation according to any one of embodiments 8 to 10, wherein the linker is selected from the group comprising a bond, a natural amino acid, an unnatural amino acid, a linear diamine, a cyclic diamine, a linear carboxylic acid, a cyclic carboxylic acid, polyethylene glycol (PEG) and combinations thereof.

12. The kit formulation according to any one of embodiments 8 to 11 wherein the linker is selected from the group comprising 4-amino-1-carboxymethylpiperidine, (R,S)-diaminoaceticacid, PEG1-24, Sar-10, 8-aminooctanoic acid, 6-aminocaproic acid, 4-(2aminoethyl)-1-carboxymethyl piperazine, diaminobutyric acid, hippuric acid, 4-amino-1-Boc-piperidine-4-carboxylic acid, Gly-aminobenzoic acid, 5-amino-3-oxa-pentyl-succinamic acid, PEG1-24-4-amino-1-carboxymethyl piperidine, Dab(shikimic acid), (D-Gln)x, (D-Asn)x.

13. The kit formulation as defined in any one of embodiments 8 to 12, wherein the linker is 4-amino-1-carboxymethyl-piperidine.

14. The kit formulation according to any one of embodiments 8 to 13, wherein said targeting moiety is selected from molecules that target VEGF receptors, analogs of bombesin or GRP receptor (GRPr) targeting molecules, molecules targeting somatostatin receptors, RGD peptides or molecules targeting αvβ3 and αvβ5, annexin V or molecules targeting the apoptotic process or molecules targeting estrogen receptors.

15. The kit formulation according to embodiment 14, wherein the GRP receptor (GRPr) targeting molecule is a GRPr targeting peptide.

16. The kit formulation according to embodiment 14, wherein said a GRP receptor (GRPr) targeting molecule comprises peptide sequences selected from the group comprising:

Gln-Trp-Ala-Val-Gly-His,
D-Phe-Gln-Trp-Ala-Val-Gly-His,
Gln-Trp-Ala-Val-Gly-His-Sta,
D-Phe-Gln-Trp-Ala-Val-Gly-His-Sta,
Gln-Trp-Ala-Val-Gly-His-Leuψ(CHOH-CH₂)-
(CH₂)₂-CH₃,
D-Phe-Gln-Trp-Ala-Val-Gly-His-Leuψ(CHOH-
CH₂)-(CH₂)₂-CH₃,
D-Phe-Gln-Trp-Ala-Val-Gly-His-Leuψ(CH₂NH),
Gln-Trp-Ala-Val-Gly-His-Leuψ(CH₂NH),
Gln-Trp-Ala-Val-Gly-His-Sta-Leu-NH₂,
or
D-Phe-Gln-Trp-Ala-Val-Gly-His-Sta-Leu-NH₂.

17. The kit formulation according to any one of embodiments 1 to 16, wherein said chelate functionalized conjugates are selected from the group comprising:
DOTA-4-amino-1-carboxymethyl-piperidine-D-Phe-Gln-Trp-Ala-Val-Gly-His-Sta-Leu-NH₂,
NOTA-4-amino-1-carboxymethyl-piperidine-D-Phe-Gln-Trp-Ala-Val-Gly-His-Sta-Leu-NH₂, or
NODAGA-4-amino-1-carboxymethyl-piperidine-D-Phe-Gln-Trp-Ala-Val-Gly-His-Sta-Leu-NH₂.

18. The kit formulation according to any one of embodiments 1 to 17, wherein said kit formulation contains 5-500 nmol of the chelate-functionalized targeting conjugate, preferably, 5-150 nmol of the chelate-functionalized targeting conjugate, more preferably, 10-100 nmol of the chelate functionalized chelate-functionalized targeting conjugate.

19. The kit formulation according to embodiment 18, wherein the formulation is a crystalline, a partially crystalline, partially amorphous or an amorphous formulation.

20. The kit formulation according to embodiment 18 or 19, wherein the lyophilized formulation is sterile.

21. The kit formulation according to any one of embodiments 1 to 20, wherein the formulation has a residual moisture of ≤1%, preferably ≤0.5%.

22. The kit formulation according to any one of embodiments 1 to 21, wherein the decomposition of the chelate-functionalized targeting conjugate in the formulations after storage for 12 months at 25° C./60% RH is ≤10%, preferably ≤5%, more preferably ≤3%.

23. The kit formulation according to any one of embodiments 1 to 22, wherein the radiochemical purity of the radiometal labeled chelate-functionalized targeting conjugate prepared from the formulation is after storage for 12 months at 25° C./60% RH≥90%, preferably ≥93%, more preferably ≥95%.

24. The kit formulation according to any one of embodiments 1 to 23, wherein the formulation for preparation of chelate-functionalized targeting conjugates is labeled with a trivalent radiometal cation or a bivalent radiometal cation.

25. The kit formulation according to any one of embodiments 1 to 24, wherein the formulation for preparation of chelate-functionalized targeting conjugates labeled with an isotope is selected from the group comprising ⁶⁸Ga, ⁶⁴Cu, ⁶⁷Cu, ¹⁷⁷Lu, ⁸⁶Y, ⁹⁰Y, ²¹²Pb, ²²⁵Ac, ²¹³Bi, ⁴⁴Sc or ¹⁸F—Al.

26. The kit formulation according to any one of embodiments 1 to 25, wherein the formulation for preparation of chelate-functionalized targeting conjugates is labeled with ⁶⁸Ga.

27. A method for preparation of a radiometal labeled chelate-functionalized targeting conjugate using said kit formulation according to any one of embodiments 1 to 26, comprising the steps of
mixing a solution of the radiometal with the kit formulation according to any one of embodiments 1 to 26,
optionally, adding at least one labeling buffer or a mixture of labeling buffers
complexing the chelate-functionalized targeting conjugate according to any one of embodiments 1 to 26 with the radiometal.

28. The method according to embodiment 27, further comprising the steps of:
adding a diluent after the step of complexation, and/or
dispensing the volume of administration of the radiometal labeled chelate-functionalized targeting conjugate.

29. The method according to embodiment 27 or 28, wherein said complexation step is performed between 0-150° C., preferably at 25° C. (room temperature) or between 50° C. and 150° C., more preferably between 80° C. and 120° C., even more preferably between 9° and 110° C.

30. The method according to any one of embodiments 27 to 29, wherein the complexation is carried out by a heating means, preferably a heater or microwave.

31. The method according to any one of embodiments 27 to 30, wherein the complexation is performed for 0.5 min to 30 min, preferably 1 min to 20 min, preferably 1 min to 10 min, more preferably 5 to 10 min.

32. The method according to any one of embodiments 27 to 31, wherein said labeling buffer is selected from the group comprising acetate buffer or formate buffer.

33. The method according to any one of embodiments 27 to 32, wherein the chelate-functionalized targeting conjugate obtained by this method exhibits a purity of ≥90%, preferably ≥92%, more preferably ≥95%.

34. The method according to any one of embodiments 27 to 33, wherein the complexation is performed at a pH value of 2.5 to 5, more preferably 3.5 to 4.5.

35. A radiometal labeled chelate-functionalized targeting conjugate obtainable by the method according to any one of embodiments 27 to 34.

36. A radiopharmaceutical composition comprising the radiometal labeled chelate-functionalized targeting conjugate according to embodiment 35.

37. A radiopharmaceutical composition comprising
the radiometal labeled chelate-functionalized targeting conjugate according to embodiment 35,
at least one non-reducing sugar selected from the group comprising trehalose and sucrose, and
at least one radio stabilizer, selected from the group comprising ascorbic acid, ascorbic acid salts, gentisic acid, gentisic acid salts, or mixtures thereof.

38. A kit comprising
a vial comprising the lyophilized kit formulation according to any one of embodiments 1 to 26;
a vial or a syringe comprising at least one labelling buffer or a mixture of labelling buffers and/or
a vial or syringe comprising a solution of the radiometal

### Examples

### Chemicals:

RM2-DOTA (GMP), ABX
Sodium acetate trihydrate
Sodium formate, Sigma Aldrich 456020-25G
Sodium L-ascorbate, Sigma, 11140-50 g
L-Ascorbic acid, (TraceSelect, Fluka, #: 05878; Sigma, #:PHR1068-2G; 20-80 mesh GMP, #0938-05, JT Baker; Roth, #: 6288.1; Appli Chem, #: 141013.1208)
2,5 Dihydroxy benzoic acid, Sigma Aldrich #: 149357
2,5 Dihydroxy benzoic acid ultra pure, Sigma Aldrich #: 39319-10×10 mg-F
Water TraceSelect, Fluka, 95305
30% HCl TraceSelect, Merck, 1.01514.0500
0.1 M HCl (1.06 ml 30% HCl dissolved up to 100 ml with water TraceSelect; Rotem Prod. #: K72001P; ABX Prod. #: HCl-103-G)
D-(+)-Trehalosedihydrate, Sigma Aldrich T9531-100G
Polyvinylpyrrolidone K25, Fluka, #: 90268
Polyvinylpyrrolidone 40, Sigma, #: PVP40
Trehalose, Fluka, #: PHR1344
Sorbitol fest, Sigma, #: PHR 1006
D-Sorbitol, Sigma-Aldrich, #: 97336-1 kg-F
Sucrose, Sigma, #: S7903
Polysorbat 80, Fluka, #: 59924-100
Polysorbat 20, Fluka, #: 44112
Mannitol, Sigma, #: PHR 1007
D-Mannitol, Sigma, #: M8429
2-Hydroxyethyl cellulose, Aldrich, #: 308633
Hypromellose, Sigma, #: H3785
Dextran, Sigma, #: D9260-50
D-(−)-Fructose, Sigma, #: F9048-100G
D-(+)-Glucose, Sigma, #: G7528-250G

### Materials:

Headspace Vial, 9.5 ml, 3131-5245-K 1 Borsilicate glass, E&Z
TC-ELU-5 Vial (15 ml)
“Vented vial adapter” von Helapet, #: IV0020
Injection vial 10R, Fiolax—clear glass, NIPRO, #: MG037-002-0049-086 (delivered by Gilyos)
10 ml clear SCHOTT (Fiolax) Type 1 Plus (SiO2) coated Vial (delivered by Adelphi)
Fluoro-Tec Septum, West Pharma, #: 13194023/50/GREY/SIL A DB (delivered by Gilyos)
Freeze Dry Stopper, FluoroTec coated, Bromobutyl 4023/50 grey, Westar RS P (delivered by Adelphi)
syringe, Injekt® F Solo, 1 ml, BBraun #: 9166017V
syringe, Injekt® F Solo, 5 mL, BBraun #: 4606051V
metal needle Sterican® Gr. 2, G 21×1/1”, 0.80×0.40, BBraun #: 4657527

### ITLCs:

Agilent Technologies, ITLC-SG Chromatography paper, cat #: SGI0001

### Equipment:

Modular Lab Pharm Tracer+heater module HRM-6299+vial adapter (3111-2603) by Eckert & Ziegler)
[⁶⁸Ga]GaC₃: Generator IGG100-50M, lot #: 1779-14, 1856-1 Eckert & Ziegler
[⁶⁸Ga]GaCP₃: Generator IGG101-50^{Gallian Pharm}, lot #: LGHE03, Eckert & Ziegler
[⁶⁸Ga]GaCl₃: Generator ID: GaG-16-151, ITG
pH-Meter 766, Calimatic, Knick
Phosphor Imager

### Analytical Methods

The stability of the peptide in the lyophilized Kit formulations was determined by analytical HPLC. The radiochemical purity of the radiometal labeled chelate-functionalized targeting conjugates was determined by HPLC and TLC.

### Analytical HPLC Method 1:

| System: | Agilent 1200 | |
|---|---|---|
| Column: | Phenomenex Onyx, C18, 100 × 4.6 mm, part#: CH0-7643 | |
| Solvent: | water + 0.1% TFA | |
| | acetonitrile + 0.1% TFA | |
| Gradient: | min | % B |
| | 00:00 | 20 |
| | 07:00 | 30 |
| | 07:20 | 100 |
| | 08:50 | 100 |
| | 09:00 | 20 |
| | 11:00 | 20 |

| | |
|---|---|
| Flow: | 2 mL/min |
| Temperature: | room temperature |
| Injection: | 10-90 μL |
| Detection: | DAD 220 nm, RAD |

Analytical HPLC method 2:

| System: | Agilent 1200 | |
|---|---|---|
| Column: | Phenomenex, Aeris Peptide XB-C18, 3.6μ, 100 × 4.6 mm, | |
| | part#: 00D-4507-E0 | |
| Solvent: | water + 0.1% TFA | |
| | acetonitrile + 0.1% TFA | |
| Gradient: | min | % B |
| | 00:00 | 23 |
| | 15:00 | 23 |
| | 15:50 | 100 |
| | 17:00 | 100 |
| | 17:50 | 23 |
| | 20:00 | 23 |

| | |
|---|---|
| Flow: | 1.5 mL/min |
| Temperature: | room temperature |
| Injection: | 10-90 μL |
| Detection: | DAD 220 nm, RAD |

TLC method: Ammonium acetate 1M: Methanol (1:1 V/V), The retention factor (Rf) specifications are as follows: Non-complexed Ga 68 species, Rf=0 to 0.1; 68Ga-RM2, Rf=0.8 to 1.

### Radiolabeling Procedure

For Eckert&Ziegler generator IGG100 or IGG 101, the generator is eluted with 5 mL 0.1 M HCl directly into the Lyophilized Kit formulation. The labeling buffer (85 mg sodium acetate in 200-300 μL traceselect water or 100 mg sodium formate in 200-300 μL) is added and the vial is heated for 8 min in a boiling water bath.

For ITG generator, the generator is eluted with 4 mL 0.05 M HCl directly into the Lyophilized Kit formulation. The labeling buffer (35 mg sodium formate in 200-300 μL) is added and the vial is heated for 8 min in a boiling water bath.

### Preparation of Lyophilized Kit Formulations

The freeze/drying cycle of the vials (fill volume 1 mL) included:
Freeze set-point −45° V, ramp rate 1° C./min
Primary/secondary dyring −30° C.-40° C., vacuum setpoint 40 mTorr,
   Overall cycle time: 48 h.

TABLE 1
Evaluated Formulations

| Formulation | Composition |
|---|---|
| Comparative | 50 μg peptide 1 |
| Formulation A | |
| Comparative | 50 μg peptide 1, 5 mg ascorbic acid |
| Formulation B | |
| Comparative | 50 μg peptide 1, 40 mg mannitol, 5 mg ascorbic acid |
| Formulation C | |
| Comparative | 50 μg peptide 1, 40 mg mannitol, 0.01% PS 80, 5 mg |
| Formulation D | ascorbic acid |
| Comparative | 50 μg peptide 1, 40 mg mannitol, 0.01% mg PS 20, 5 mg |
| Formulation E | ascorbic acid |
| Formulation 1a | 50 μg peptide 1, 30 mg trehalose, 5 mg ascorbic acid |
| Formulation 1b | 50 μg peptide 1, 50 mg trehalose, 5 mg ascorbic acid |
| Formulation 1c | 50 μg peptide 1, 100 mg trehalose, 5 mg ascorbic acid |
| Formulation 1d | 50 μg peptide 1, 50 mg trehalose, 2 mg ascorbic acid |
| Formulation 1e | 50 μg peptide 1, 50 mg trehalose, 10 mg ascorbic acid |
| Formulation 2 | 50 μg peptide 1, 50 mg trehalose, 5 mg sodium ascorbate |
| Formulation 3 | 50 μg peptide 1, 50 mg trehalose, 15 mg gentisic acid |
| Formulation 4 | 50 μg peptide 2, 50 mg sucrose, 5 mg ascorbic acid |
| Formulation 5 | 50 μg peptide 1, 40 mg trehalose, 10 mg dextran, 5 mg |
| | ascorbic acid |

### Evaluation of the Appearance and the Residual Moisture of the Lyophilized Kit Formulations

TABLE 2
Characteristics of Lyophilized Formulations

| | | Residual |
|---|---|---|
| Formulation | Cake Appearance | moisture (%) |
| Comparative Formulation A | − (white droplets) | nd |
| Comparative Formulation B | − (total collapse) | nd |
| Comparative Formulation C | +++ (intact structure) | 0.399 ± 0.033 |
| Comparative Formulation D | +++ (intact structure) | 0.437 ± 0.044 |
| Comparative Formulation E | +++ (intact structure) | 0.615 ± 0.029 |
| Formulation 1a | ++ (intact structure, | 0.265 ± 0.041 |
| | weak shrinkage) | |
| Formulation 1b | ++ (intact structure, | 0.314 ± 0.091 |
| | weak shrinkage) | |
| Formulation 1c | ++ (intact structure, | 0.166 ± 0.045 |
| | weak shrinkage) | |
| Formulation 1d | ++ (intact structure, | 0.272 ± 0.032 |
| | weak shrinkage) | |
| Formulation 1e | ++ (intact structure, | 0.376 ± 0.070 |
| | weak shrinkage) | |
| Formulation 2 | ++ (intact structure, | nd |
| | weak shrinkage) | |
| Formulation 3 | ++ (intact structure, | nd |
| | weak shrinkage) | |
| Formulation 4 | ++ (intact structure, | 0.136 ± 0.003 |
| | weak shrinkage) | |
| Formulation 5 | ++ (intact structure, | nd |
| | weak shrinkage) | |

nd not determined

### Evaluation of the Peptide Stability in the Lyophilized Kit Formulations

The lyophilized Kit Formulations have been stored at room temperature or 40° C./60% relative humidity. The stability of the peptide was tested at baseline and a several time points by analytical HPLC (Table 3 and Table 4).

Kit formulations with mannitol (Comparative Formulations C, D, E) led to a significant decomposition of the peptide during storage at room temperature as well as at 40° C. No or only minor peptide degradation was observed for the Kit Formulations containing the non-reducing sugars trehalose or sucrose.

TABLE 3
Stability of Lyophilized Formulations at RT

| | Peptide decomposition (%) | | | |
|---|---|---|---|---|
| | Month | Month | Month | Month |
| Formulation | 1 | 3 | 6 | 12 |
| Comparative Formulation A | 1 | 1 | 1 | 1 |
| Comparative Formulation B | 4 | 4 | nd | 10 |
| Comparative Formulation C | 4 | 20 | 33 | 40 |
| Comparative Formulation D | 3 | 18 | 30 | 35 |
| Comparative Formulation E | 4 | 15 | 32 | 36 |
| Formulation 1a | 1 | 1 | 3 | 3 |
| Formulation 1b | 1 | 1 | 1 | 2 |
| Formulation 1c | 0 | 1 | 1 | 1 |
| Formulation 1d | 0 | 0 | 2 | 1 |
| Formulation 1e | 1 | 0 | 2 | 0 |
| Formulation 2 | 3 | 0 | 0 | 0 |
| Formulation 3 | 11 | 6 | 11 | 16 |
| Formulation 4 | 0 | 0 | 0 | 1 |
| Formulation 5 | 1 | 0 | 3 | 1 |

nd not determined

TABLE 4
Stability of Lyophilized Formulations at 40° C.

| | Peptide decomposition (%) | | |
|---|---|---|---|
| Formulation | Week 2 | Week 6 | Week 12 |
| Comparative Formulation A | 0 | 0 | 1 |
| Comparative Formulation B | 3 | 10 | 51 |
| Comparative Formulation C | 28 | 58 | 62 |
| Comparative Formulation D | 20 | 55 | 59 |
| Comparative Formulation E | 26 | 55 | 59 |
| Formulation 1a | 1 | 1 | 1 |
| Formulation 1b | 1 | 1 | 1 |
| Formulation 1c | 0 | 2 | 0 |
| Formulation 1d | 1 | 0 | 1 |
| Formulation 1e | 0 | 0 | 1 |
| 1Formulation 2 | 0 | 0 | 0 |
| Formulation 3 | 0 | 11 | nd |
| Formulation 4 | 0 | 1 | 0 |
| Formulation 5 | 0 | 0 | 1 |

nd not determined

Evaluation of Radiochemical Purity Obtained after Storage of the Kit Formulations

TABLE 5
Radiolabeling of Lyophilized Formulations

| | Radiochemical Purity after radiolabeling of Kits stored | | | | |
|---|---|---|---|---|---|
| | | 3 | 3 | 6 | 12 |
| | 0 | months | months | months | months |
| Formulation | months | (40° C.) | (RT) | (RT) | (RT) |
| Comparative | 96 | nd | 96 | 95 | 98 |
| Formulation A | | | | | |
| Comparative | 93 | nd | 90 | 91 | 95 |
| Formulation B | | | | | |
| Comparative | 91 | 65 | 71 | 69 | nd |
| Formulation C | | | | | |
| Comparative | 92 | 70 | 77 | 71 | nd |
| Formulation D | | | | | |
| Comparative | 93 | 63 | 74 | 67 | nd |
| Formulation E | | | | | |
| Formulation 1a | 97 | nd | 96 | 96 | 96 |
| Formulation 1b | 96 | 96 | 96 | 96 | 97 |
| Formulation 1c | 97 | 97 | 97 | 98 | nd |
| Formulation 1d | 95 | 98 | 97 | 97 | 96 |
| Formulation 1e | 93 | 95 | 95 | 96 | 97 |
| Formulation 2 | 96 | nd | 94 | 95 | 90 |
| Formulation 3 | 97 | nd | 94 | 94 | 88 |
| Formulation 4 | 97 | 97 | 98 | 98 | 98 |
| Formulation 5 | 91 | nd | 90 | 90 | 85 |

nd not determined

The invention claimed is:

1. A lyophilized kit formulation for the preparation of radiometal labeled chelate-functionalized targeting conjugates comprising
a chelate-functionalized targeting conjugate comprising
i. a chelating moiety
ii. at least one targeting moiety, wherein said targeting moiety is a GRP receptor (GRPr) targeting peptide, and
iii. optionally, at least one linker, connecting the chelating moiety with the GRP receptor (GRPr) targeting moiety,
at least one non-reducing sugar selected from the group comprising trehalose and sucrose, and
at least one radio stabilizer, selected from the group comprising ascorbic acid, ascorbic acid salts, gentisic acid, gentisic acid salts, or mixtures thereof.

2. The kit formulation according to claim 1, wherein the concentration of the non-reducing sugar in said formulation is 10-600 μmol, preferably, 20-500 μmol, more preferably, 50-300 μmol.

3. The kit formulation according to claim 1, wherein the concentration of said radiostabilizer in the formulation is 1-500 μmol, preferably 5-250 μmol, more preferably 10-100 μmol.

4. The kit formulation according to claim 1, wherein the amount of ascorbic acid in the formulation is 1-20 mg, more preferably 1-10 mg.

5. The kit formulation according to claim 1, wherein said chelating moiety is selected from the group comprising TACN, TACN-TM, DTAC, H3NOKA, NODASA, NODAGA, NOTP, NOTPME, PrP9, TRAP, Trappist Pr, NOPO, TETA; Tris(hydroxypyridinone) (THP) and derivatives, chelates open chain such as HBED, DFO or desferrioxamine or desferal, EDTA, 6SS, B6SS, PLED, TAME, YM103; NTP (PRHP)3; the H2dedpa and its derivatives such as H2dedpa-1, 2-H2dedpa, H2dp-bb-NCS, and H2dp-N-NCS; (4,6-Me02sal) 2-BAPEN; and citrate and derivatives thereof.

6. The kit formulation according to claim 1, wherein the linker is selected from the group comprising a bond, a natural amino acid, an unnatural amino acid, a linear diamine, a cyclic diamine, a linear carboxylic acid, a cyclic carboxylic acid, polyethylene glycol (PEG) and combinations thereof.

7. The kit formulation according to claim 1, wherein said a GRP receptor (GRPr) targeting molecule comprises peptide sequences selected from the group comprising:
Gln-Trp-Ala-Val-Gly-His,
D-Phe-Gln-Trp-Ala-Val-Gly-His,
Gln-Trp-Ala-Val-Gly-His-Sta,
D-Phe-Gln-Trp-Ala-Val-Gly-His-Sta,
Gln-Trp-Ala-Val-Gly-His-Leuψ(CHOH-CH₂)-
(CH₂)₂-CH₃,
D-Phe-Gln-Trp-Ala-Val-Gly-His-Leuψ(CHOH-
CH₂)-(CH₂)₂-CH₃,
D-Phe-Gln-Trp-Ala-Val-Gly-His-Leuψ(CH₂NH),
Gln-Trp-Ala-Val-Gly-His-Leuψ(CH₂NH),
Gln-Trp-Ala-Val-Gly-His-Sta-Leu-NH₂,
or
D-Phe-Gln-Trp-Ala-Val-Gly-His-Sta-Leu-NH₂.

8. The kit formulation according to claim 1, wherein the formulation for preparation of chelate-functionalized GRP receptor targeting conjugates is labeled with a trivalent radiometal cation or a bivalent radiometal cation.

9. A method for preparation of a radiometal labeled chelate-functionalized GRP receptor targeting conjugate using said kit formulation according to claim 1, comprising the steps of
mixing a solution of the radiometal with the kit formulation according to claim 1,
optionally, adding at least one labeling buffer or a mixture of labeling buffers, and
complexing the chelate-functionalized GRP receptor targeting conjugate according to claim 1 with the radiometal.

10. A radiometal labeled chelate-functionalized targeting conjugate obtainable by the method according to claim 9.

11. A radiopharmaceutical composition comprising the radiometal labeled chelate-functionalized GRP receptor targeting conjugate according to claim 10.

12. A kit comprising
a vial comprising the lyophilized kit formulation according to claim 1;
a vial or a syringe comprising at least one labelling buffer or a mixture of labelling buffers and/or
a vial or syringe comprising a solution of the radiometal.
